(19) United States
(12) Patent Application Publication
(10) Pub. No.: US 2008/0260579 A1
(43) Pub. Date: Oct. 23, 2008

(54) Sheet-like material with hydrophilic and hydrophobic egions and their production

(75) Inventors: INGO NEUBERT, Norderstedt (DE); MAREN KLOSE, Seevetal (DE)

Correspondence Address: NORRIS, MCLAUGHLIN & MARCUS, PA, 875 THIRD AVENUE, 18TH FLOOR, NEW YORK, NY, 10022, US

(73) Assignee: TESA AKTIENGESELLSCHAFT, Hamburg (DE)

(21) Appl. No.: 11/774,086

(22) Filed: Jul. 6, 2007

(30) Foreign Application Priority Data
Apr. 17, 2007 (DE) 10 2007 018 383.8

(51) Int. Cl. G01N 33/48; B32B 5/00

(52) U.S. Cl. 422/60; 422/56; 422/57; 428/327

(57) ABSTRACT

Sheet-like material in particular for use in biosensors and diagnostic strips composed of a substrate material on whose upper side at least one hydrophilic region and at least one hydrophobic region are present and arranged alongside one another, where
the contact angle of the hydrophilic region with water is smaller than 30° and
the contact angle of the hydrophobic region with water is greater than 90°.

The present invention relates to a sheet-like material which, by virtue of hydrophilic and hydrophobic regions, controls the transport of biological liquids, such as blood, urine, saliva or cell fluid, in biosensors or in analytical test strips.

In modern medical diagnostics, the numb ps used, known as diagnostic test strips, and the number of biosensors used, er of analytical test striis constantly increasing. These applications are generally known as microfluidic devices and lab-on-a-chip application. Biosensors can, for example, be used on biological liquids firstly to search for pathogens, incompatibilities, DNA activity or enzyme activity, and secondly to determine content of glucose, cholesterol, proteins, ketones, phenylalanine or enzymes.

Detector reactions or reaction cascades take place on the biosensors or test strips. For this, the biological test liquid has to be transported to the reaction site or to various reaction sites. Modern biosensors and test strips are therefore composed of at least one microchannel or microchannel system, through which the test liquid is transported. The height and width of the microchannels are typically from 5 to 1500 μm. Transport within the channels takes place via capillary or centrifugal forces. The results of the detector reactions are mostly read off optically or electrochemically.

Examples that may be mentioned of typical applications are microfluidic devices (e.g. US 2002/0112961 A1, U.S. Pat. No. 6,601,613B2, U.S. Pat. No. 7,125,711 B2, EP 1 525 916 A1), biosensors (for example DE 102 34 564 A1, U.S. Pat. No. 5,759,364 A1) and blood sugar test strips (for example WO 2005/033698 A1, U.S. Pat. No. 5,997,817 A1). Other examples of applications are DNA microarrays and immunoassays.

In all of these systems, it is important to influence liquid transport. Firstly, very rapid liquid transport is desirable for reasons of short test times. Secondly, a delay in, or a stoppage of, liquid transport is necessary in order, for example, to permit the detector reaction to take place under stationary-state conditions.

Various investigations on the topics of capillary action and transport of liquids in capillaries can be found in the literature. The capillary pressure and the ascension of a liquid column in a capillary depend on the surface tension of the liquid, the viscosity of the liquid, the angle of wetting and the diameter of the capillary. The following formula is used to determine ascension (equation 1 (eq. 1)):

$$h = \frac{4 * {γ}_{l} * cos θ}{g ( {ζ}_{l} - {ζ}_{g} ) * d} \\ \begin{matrix}h & - & ascension or depression \\ {γ}_{l} & - & surface tension of liquid \\ {ζ}_{l} & - & density of liquid \\ {ζ}_{g} & - & density of gas ( air ) \\ g & - & acceleration due to gravity \\ θ & - & contact angle ( wetting angle ) \\ d & - & internal diameter of capillary\end{matrix} \qquad eq . 1$$

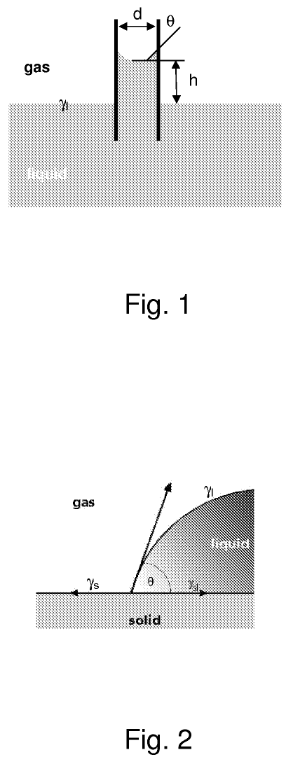

FIG. 1 illustrates equation 1.

From this it is apparent that capillary forces increase as capillary diameter decreases. Flow rate in a capillary can therefore be reduced by enlarging the cross section of a microchannel. Another important parameter affecting flow rate of a given liquid is the surface tension of the inner side of the channel, whereas viscosity is a parameter that cannot be varied for a given liquid.

If the wetting angle between liquid and capillary wall is very small, capillary ascension occurs, meaning that the liquid rises in the capillary. However, if the contact angle is >90° the result is capillary depression, and the surface of the liquid in the capillary is below the surface of the liquid outside (W. Bohl “Technische Strömungslehre” [Rheology], 13th revised and extended edition, Vogel Verlag, June 2005, ISBN: 3834330299, pages 37 et seq.).

Numerous investigations of surface tension and of the phenomenon of wettability of solids are found in the literature.

Young's equation (eq. 2) describes the wetting of a solid by a liquid (in which connection see FIG. 2):

$${γ}_{l} * cos θ = {γ}_{s} - {γ}_{sl} \\ \begin{matrix}θ & - & contact angle ( wetting angle ) \\ {γ}_{l} & - & surface tension of liquid \\ {γ}_{s} & - & surface tension of solid \\ {γ}_{sl} & - & interfacial tension between liquid and solid\end{matrix} \qquad eq . 2$$

If the surface tensions of the solid and of the liquid are markedly different, the result is a contact angle θ>>90°. The surface of the solid cannot be wetted by the liquid. In the range from 90° to 20°, wetting of the surface of the solid occurs. At contact angles θ<20°, the surface tensions are very similar between liquid and solid, and very good wetting of the surface of the solid by the liquid occurs. For contact angles θ<<20° (θ˜0°) the liquid spreads on the surface of the solid (see “Die Tenside” [Surfactants], Kosswig/Stache, Carl Hanser Verlag, 1993, ISBN 3-446-16201-1; page 23).

The literature describes the use of surfactants, which the person skilled in the art knows are substances active at interfaces, for improvement of wettability. Surfactants are molecules or polymers which are composed of a non-polar/hydrophobic portion (tail) and a polar/hydrophilic group (head). To improve wettability of surfaces, surfactants are added to the aqueous liquid. The surfactant reduces the surface tension of the aqueous liquid at the interfaces (liquid-solid and liquid-gaseous). This effect of improvement of wettability of surfaces is measurable in a reduction of the contact angle and in reduction of the surface tension of the liquid. The person skilled in the art makes a distinction between anionic, cationic, amphoteric and nonionic surfactants. The hydrophobic tail of surfactants can be composed of linear or branched alkyl, alkylbenzyl, perfluorinated alkyl or siloxane groups. Possible hydrophilic head groups are anionic salts of carboxylic acids, of phosphoric acids, of phosphonic acids, of sulphates, or of sulphonic acids, or are cationic ammonium salts or nonionic polyglycosides, polyamines, polyglycol esters, polyglycol ethers, polyglycolamines, polyfunctional alcohols or alcohol ethoxylates (see also Ullmann's Encyclopedia of Industrial Chemistry, Vol. A25, 1994, page 747).

In principle, better wettability of the inner side of the microchannels can lead to an increase in transport velocity for the biological liquid within the microchannels. An increase in surface tension and therefore better wettability can be achieved via hydrophilic coatings with polar polymers, for example polyvinylpyrrolidone, polycaprolactam, polyethylene glycol or polyvinyl alcohol. However, the wettability or hydrophilic properties of these coatings are often inadequate for rapid transport of biological liquids in microchannels.

Another possibility consists in chemical or physical modification of the surfaces. Standard methods for this are corona treatment and flame treatment. However, these treatments are not stable over time. The markedly increased surface energy resulting from the surface treatment decreases to the initial value after just a few days.

An increase in the level of hydrophilic properties is likewise achieved via etching of the surface with a strong acid. Surface etching of industrial foils is achieved by using, for example, oxidizing acids, such as chromic-sulphuric acid mixture, or potassium permanganate in combination with sulphuric acid. Polyester foils (PET) are hydrolyzed in industry, usually via chemical treatment with, for example, trichloroacetic acid or potassium hydroxide on the surface, as disclosed in WO 2005/111606 A1. These methods give wettability or surface tension which remains stable after storage. However, the wetting properties are not homogeneous across the surface.

The surface can moreover be modified via plasma treatment. To this end, the surface is treated with a plasma in vacuo. Introduction of gases or organic substances can adjust the surface properties as desired. For example, either hydrophilic or hydrophobic layers can be produced on the surface. U.S. Pat. No. 6,955,738 B2 describes the application of the said process.

WO 01/02093 A2 describes the use of a foil with a microstructured surface for the control of liquid transport.

DE 102 34 564 A1 describes a biosensor which is composed of a planar sensor or test strip and of a compartmented reaction- and test-chamber superstructure, produced via embossing of a PVC foil. For transport of the biological liquid, the channel that receives the specimen, and the measurement chamber, are equipped with a hydrophilic web or a surfactant. A very similar electrochemical sensor is described in U.S. Pat. No. 5,759,364 A1. The sensor is composed of a printed base plate and of an embossed top foil composed of PET or polycarbonate. The test chamber here has a coating of a polyurethane ionomer for accelerating liquid transport.

A number of publications mention the use of hydrophilic materials such as webs (DE 30 21 166 A1), membranes (DE 198 49 008 A1) and foils (EP 1 358 896 A1, WO 01/67099 A1), but without any more detailed characterization of the hydrophilic coatings.

DE 198 15 684 A1 describes an analytical aid composed of a capillary-action zone, a stamped-out piece of adhesive tape, and a capillary-action top foil. The capillary-action top foil has hydrophilic surface properties, which are achieved by aluminium-metallization of the top foil and subsequent oxidation.

US 2005/0084681 A1 discloses a surface with a hydrophilic coating. This coating is composed of a surfactant, preferably a nonionic alcohol ethoxylate, and a stabilizer, preferably an alkylbenzenesulphonate.

EP 1 647 568 A1 describes what is known as an antifog coating. This coating is applied to a polyester foil and serves to avoid water droplet formation in food packaging. The antifog coating involves a hydrophilic coating composed of an anionic surfactant, a polyvinylpyrrolidone as matrix polymer, and water.

Hydrophilic foils for use in medical diagnostic strips have now become commercially available, examples being the products 9962 and 9971 from 3M Inc., the use of which is indicated in US 2002/0110486 A1 and EP 1 394 535 A1. These products have a polyester foil equipped either on one side or on both sides with a hydrophilic coating. This coating is composed of a polyvinylidene chloride coating which comprises a surfactant based on an alkylbenzylsulphonate. The surfactant must first migrate to the surface of the coating before the hydrophilic surface properties can be developed. A detailed investigation shows that although these products are suitable for the transport of biological liquids in diagnostic strips they have considerable shortcomings in relation to homogeneity, transport velocity and ageing resistance.

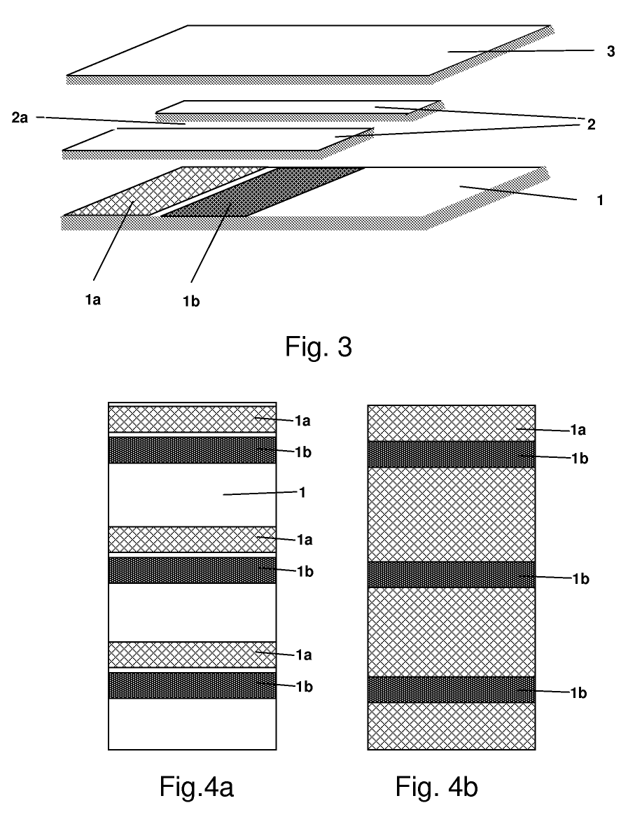

Other products likewise commercially available are the hydrophilic foils ARflow® 90128 and ARflow® 90469 from Adhesives Research Inc., the use of which is indicated in U.S. Pat. No. 5,997,817 A1. These products are composed of a polyester foil coated with a thermoplastic copolyester, with addition of a surfactant. The principle of action is analogous to that of the products described above from 3M Inc. To avoid inhomogeneity, considerably larger amounts are added here: from about 5 to 8% of the surfactant. However, a consequence of this is that the surface of the hydrophilic coating is waxy. Adequate adhesive bond strength to pressure-sensitive adhesive tapes (see layer 2 in FIG. 3) cannot be achieved on this coating. This often leads to delamination of the composite in the production process for test strips.

The literature likewise discloses some examples in which hydrophilic and hydrophobic regions are utilized specifically for control of liquid transport. In U.S. Pat. No. 6,601,613 B2, capillary geometry is used to control liquid transport. Another possibility mentioned, but not described in any further detail, is the use of surfactants or hydrophobic polymers.

U.S. Pat. No. 6,969,166 B2 describes a modified surface with two different contact angles. The modification is achieved via digital print (inkjet print) of hydrophobic polymers based on fluorine polymers or on silicone polymers, and, respectively, of hydrophilic polymers. The static contact angles given in the examples for the hydrophilic coating are 75°.

It is an object of the present invention to provide a sheet-like material with hydrophilic and hydrophobic regions which is suitable to meet the requirements for use in biosensors and diagnostic test strips and for construction of the same, and which specifically influences in a defined manner the transport of the biological liquid in the test channels. The intention here is moreover to ensure that the properties, and specifically the wetting properties and transport properties, of the sheet-like material are retained even after a long storage time.

This object is achieved via a sheet-like material as set out in the main claim. The subclaims provide advantageous embodiments of the inventive subject matter. The invention moreover encompasses the possible use of the inventive foil inter alia in medical sensors or diagnostic strips for investigation of biological liquids.

Accordingly, the invention provides a sheet-like material composed of a substrate material on whose upper side at least one hydrophilic region and at least one hydrophobic region are present and arranged alongside one another, where
the contact angle of the hydrophilic region with water is smaller than 30° and
the contact angle of the hydrophobic region with water is greater than 90°.

In one advantageous embodiment of the invention, on the substrate material a plurality of hydrophilic and a plurality of hydrophobic regions are present arranged alongside one another, in particular in alternation alongside one another. It is further preferable that they have mutually contacting boundary lines.

The hydrophilic and hydrophobic regions on the upper side of the substrate material can be produced by application of the regions alongside one another or by partial application, to a full-surface coating of one of the regions, of the other region(s).

The hydrophilic regions are preferably composed of a coating which has a surface tension of at least 63 mN/m and a contact angle with water smaller than 30°, preferably smaller than 25°, and which preferably comprises at least one surfactant. The coating is particularly preferably composed of at least one surfactant and of at least one polymer.

The hydrophobic regions are preferably composed of a coating which has a surface tension of at most 30 mN/m, a contact angle with water greater than 90°, preferably greater than 95°, and a separation value smaller than 50 cN/cm in a test using tesa® 7475 adhesive tape. The coating is particularly preferably composed of a release lacquer based on a stearyl compound, in particular on a stearylcarbamate, on a fluoropolymer or on a silicone polymer. The hydrophobic coating can preferably be crosslinked by means of UV radiation.

Surprisingly, transport of the test liquid can be controlled very effectively when the inventive material is used as top foil or as wall of a transport channel or of a reaction channel in a biosensor or in an analytical test strip. It is surprising to the person skilled in the art that, in a channel as shown in FIG. 3, firstly the hydrophilic regions considerably accelerate liquid transport, and secondly the hydrophobic regions can stop transport of the test liquid completely, irrespective of the geometry of the capillary. These results are unforeseeable, because of the great complexity of the parameters affecting capillary forces, e.g. geometry of the capillary, differences in surface tension of the various walls, differences in polarity and viscosity of the biological test liquid, and also the effect of surfactants.

FIG. 3 shows an example of the structure of a diagnostic strip with a microchannel 2a, in this case formed via a stamped-out section of a double-sided pressure-sensitive adhesive tape 2. However, the microchannel 2a can also be produced via other processes, e.g. microembossing, injection moulding, laser processes, lithographic processes or sandblasting. The microchannel 2a formed from the pressure-sensitive adhesive tape 2 has been adhesive-bonded to the base film 3 on one side. This base film 3 therefore forms one wall of the microchannel and can also have functional layers, e.g. electrical conductor tracks or layers with detector reagents or with enzymes. A further wall which seals the microchannel 2a is the upper side of the inventive material 1, where this side has a hydrophilic region 1a and a hydrophobic region lb. The hydrophilic region 1a here has been positioned at the opening of the microchannel 2a, thus permitting the start of liquid transport into the channel.

FIGS. 4a and 4b show preferred embodiments of the upper sides of the inventive web-like material 1. FIG. 4a shows the upper side of a sheet-like material 1, where this side has hydrophilic regions 1a and hydrophobic regions 1b in the form of strip-like coatings, the regions not having mutual contact. FIG. 4b shows the upper side of a sheet-like material, where this side has a full-surface hydrophilic coating 1a, to which the hydrophobic regions 1b have been partially applied in the form of strips.

Surfactants that can be used are compounds composed of linear or branched alkyl, alkylbenzyl, perfluorinated alkyl or siloxane groups, having hydrophilic head groups, e.g. anionic salts of carboxylic acids, of phosphoric acids, of phosphonic acids, or of sulphates, or sulphonic acids, or sulphosuccinic acid, or cationic ammonium salts or norlionic polyglycosides, polyamines, polyglycol esters, polyglycol ethers, polyglycolamines, polyfunctional alcohols or alcohol ethoxylates. This selection is a list of examples and does not restrict the inventive concept to the surfactants mentioned.

The following suitable surfactants may be mentioned by way of example:
nonionic fatty alcohol ethoxylate surfactants, for example Tego Surten® W111 from Degussa AG or Triton® X-100 and Tergitol® 15-S from Dow Chemicals Inc
nonionic fluorosurfactants, for example Fluorad® FC-4430 and FC-4432 from 3M Inc., Zonyl® FSO-100 from DuPont Inc. and Licowet® F 40 from Clariant AG
nonionic silicone surfactants for example Q2-5211 and Sylgard® 309 from Dow Corning Inc., Lambent® 703 from Lambent Technologie Inc. and Tegopren® 5840 from Degussa AG
ionic alkyl sulphate salt, for example Rewopol® NLS 28 from Goldschmidt GmbH
ionic sulphosuccinic salts, for example Lutensit® A-BO from BASF AG, Aerosol® OT-NV from Cytec Industries Inc or Rewopol® SB DO 75 from Goldschmidt GmbH

It is preferable that the hydrophilic coating is composed—as mentioned above—of one of the surfactants mentioned above by way of example and of at least one polymer. The polymer serves here as binder for the coating comprising surfactant. As polymer it is possible to use any of the known film-forming binders from the printing ink industry. As binder it is advantageous to use a polymer having polar functional groups, for example hydroxy, carboxy, ether, ester, amine, or amide groups. By way of non-restricting example, suitable binders that may be mentioned are homo- or copolymers, such as polyvinylpyrrolidone, polyvinyl butyral, polyester, polyacrylate, polyacrylic acid, polyvinyl acetate, polyvinyl alcohol, polyacrylamide, polyamide, polyethylene glycol, polypropylene glycol, cellulose derivatives.

The hydrophilic coating can moreover likewise comprise organic dyes or inorganic pigments, antioxidants and/or fillers (in which connection see “Plastics Additives Handbook”, Chapter on “Antioxidants”, “Colorants”, “Fillers”, Carl Hanser Verlag, 5th Edition).

The hydrophobic coating is preferably composed of a release lacquer. Typical release lacquer coatings are those produced from stearyl compounds, from fluoropolymers or from silicone polymers. These release lacquer coatings feature hydrophobic character or low surface tension. Fluorinated polymers or polymers based on polysiloxane are particularly suitable, alongside the use of stearyl carbamate as inventive hydrophobic coating. Examples of companies producing polysiloxane release lacquer coatings are Wacker, Rhodia or Dow Corning. Coatings based on solvent or on emulsion are suitable, as also are 100% systems. These polysiloxane coatings are usually crosslinked via a free-radical reaction, addition reaction or condensation reaction. The crosslinking takes place either thermally during the drying of the coating or particularly preferably via UV radiation applied to a 100% system. An example that may be mentioned is the UV silicone system Syl-Off UV® from Dow Corning and UV-crosslinking printable release lacquers, e.g. UVX00192, UAAS0032 or UAS00107 from XSys GmbH. A release lacquer based on fluorinated polymers is likewise suitable for the hydrophobic coating. Examples that may be mentioned are coatings composed of polymers or copolymers composed of vinylidene fluoride, hexafluoropropene, hexafluoroisobutylene and tetrafluoroethene.

The hydrophobic coating can likewise comprise organic dyes or inorganic pigments, antioxidants and/or fillers (in which connection, see “Plastics Additives Handbook”, Chapter on “Antioxidants”, “Colorants”, “Fillers”, Carl Hanser Verlag, 5th Edition).

The inventive substrate material is based on substrate materials which are conventional and familiar to the person skilled in the art, e.g. foils composed of polyethylene, polypropylene, oriented polypropylene, polyvinyl chloride, polyester and particularly preferably polyethylene terephthalate (PET). Monofoils or coextruders or laminated foils can be involved here and these can be unoriented or monoaxially or biaxially oriented foils. This is a list of examples and is not comprehensive. The surface of the foils can have been microstructured via suitable processes, such as embossing or etching, or lasers. It is also possible to use laminates, nonwovens, textiles or membranes. The substrate materials can have been chemically or physically pretreated by the standard methods to improve anchoring of the coating, and an example that may be mentioned is corona treatment or flame treatment. Priming of the substrate material is also possible to promote adhesion, for example using PVC, PVDC, or thermoplastic polyester copolymers. The thickness of the substrate foil is from 12 to 350 μm, preferably from 50 to 150 μm.

The form in which the hydrophilic and/or the hydrophobic region has been applied to the substrate material can be that of a full-surface or partial coating using a solvent. Solvents used comprise water, alcohols, ethanol, or higher-boiling-point alcohols, such as n-butanol or ethoxyethanol, ketones, such as butanone, esters, such as ethyl acetate, alkanes, such as hexane or toluene, or a mixture composed of the abovementioned solvents. The usual coating processes can be used for coating. Examples that may be mentioned are spray coating, halftone-roll application, Meyer-Bar coating, multiroll-application coating, printing processes, such as screen printing or flexographic printing, and condensation coating. The hydrophilic and, respectively, hydrophobic properties can also be modified by coating in the form of separate points in a grid. This can be used to control the surface tension of the regions via the distance between, and size of, the individual points in the grid. A typical process for producing halftone prints is screen printing or digital printing, for example inkjet printing. However, the preferred embodiment is composed of hydrophilic and hydrophobic regions which form a coherent film.

In one preferred embodiment, the form in which the hydrophilic and hydrophobic regions are applied by a printing process to a biaxially oriented PET foil is that of two parallel lines whose line width is from 0.2 to 4.0 mm.

The inventive material can advantageously be used in medical applications, preferably in diagnostic strips, biosensors, point-of-care devices, or a microfluidic device, these being used to investigate biological liquids.

The material is particularly suitable in biosensors or analytical test strips, the arrangement of the material here being such that the upper side of the substrate material forms the wall of a transport channel or of a reaction channel.

### Test methods

### Surface Tension and Contact Angle Measurement

Contact angle with water and surface tension on solid surfaces are measured to EN 828:1997 by a G2/G402 device from Kruss GmbH. Surface tension is determined by the Owens-Wendt-Rabel & Kaeble method, the contact angle having been measured using deionized water and diiodomethane. In each case, the values are obtained by taking the average of four measured values.

### Channel Test

### Function Test

To assess the transport behaviour of an aqueous test liquid, a capillary test is carried out. For this, a stamped-out section of a double-sided adhesive tape whose thickness is 80 μm (tesa® 4980, a double-sided pressure-sensitive adhesive tape composed of a 12 μm PET substrate foil coated on both sides with an acrylate adhesive mass (in each case 34 g/m²), product thickness 80 μm) is laminated to a PET foil of thickness 100 μm (Hostaphan® RN 100 from Mitsubishi Polyesterfilm GmbH). The stamped-out section forms a test channel whose width is 1 mm and whose length is 3 cm. The channel is open at both ends. This test channel is then covered with the foil to be tested, so that the surface to be tested forms one wall of the channel. The regions here are positioned so that the hydrophilic region is at the margin of the channel and the hydrophobic region is in the interior of the channel. The dimensions of the channel or the capillary are: height 75 μm and width 1 mm (see FIG. 3).

The test channel is held with the aperture at whose end the hydrophilic region has been positioned in a test liquid composed of deionized water and 1% by weight of naphthol red. Transport of the test liquid in the hydrophilic region is observed by means of a video camera, as also is the stoppage of transport when the hydrophobic region is reached. The test channel is left for some further minutes in the test liquid in order to ensure that passage of time does not cause onward transport of the liquid within the hydrophobic region.

The channel test is also to be carried out after the channels or foils to be tested have been stored at 23° C., 40° C. and 70° C., in order to check ageing resistance and storage stability.

Biological liquids, such as blood, are likewise used as test liquid, but these are less appropriate, since they are subject to variations in properties. For example, the viscosity of blood varies very markedly. The viscosity of blood depends on the haematocrit value.

### Separation Forces

This test determines the release behaviour of release lacquer coatings using the standard test adhesive tape tesa® 7475.

Characteristics/of tesa® 7475 single-sided adhesive tape used in the test

| | | | Thickness | |
|---|---|---|---|---|
| | Adhesive | Protective | (without | |
| Substrate | mass | layer | protective layer) | Adhesion/steel |
| PVC foil | 95 g/m² | Release | 0.135 mm | 12.5 N/cm |
| 40 μm, white | acrylate | paper | | |

To determine the separation forces, strips of width 20 mm of the tesa® 7475 adhesive tape used in the test are adhesive-bonded to the release lacquer coating to be tested. The sample is then stored for 24 h at 70° C. under a block weighing 20 kg. After conditioning for 2 h at 23° C. and 50% relative humidity, a tensile tester with separation velocity of 0.3 m/min is used to determine the force needed to peel the adhesive tape at an angle of 180° C. from the release lacquer coating. The method of measurement here is based on PSTC-4B.

A number of examples will be used below to provide further illustration of the invention, but they are not intended to restrict the invention unnecessarily.

### EXAMPLES

### Inventive Example 1

Hostaphan® RN 100 PET film from Mitsubishi Polyesterfilm GmbH of thickness 100 μm is corona-pretreated on one side and then coated with a solution composed of 5% by weight of Rewopol® SB DO 75 (sodium diisooctyl sulfosuccinate) from Goldschmidt GmbH in ethanol, by means of a halftone roll. The coating is dried in a drying tunnel at 120° C.

In a second operation, this hydrophilic coating is printed with a hydrophobic line of width 1.2 mm, by the flexographic process. The hydrophobic coating used comprises the flexographic lacquer UVF00080 (UV-drying, free-radical-crosslinking flexographic lacquer) from XSys GmbH with 5% of UAS00107 (UV-drying, free-radical-crosslinking silicone additive). The coating is cured by means of UV radiation.

The hydrophilic coating exhibits very good wetting properties and high transport velocity of the aqueous test liquid in the test channel. Liquid transport in the test channel stops at the edge of the hydrophobic line. No transport over the edge of the hydrophobic line takes place even after further supply of test liquid. Nor does this transport behavior of the test liquid alter when the test channels are stored for 6 weeks at 40° C. or 70° C. prior to the test.

### Inventive Example 2

The corona-pretreated Hostaphan® RN 100 PET film from Mitsubishi Polyesterfilm GmbH of thickness 100 μm is printed by the flexographic printing process first with a hydrophobic line of width 2.0 mm and then with a hydrophobic line of width 1.0 mm. The two lines run parallel to one another and the distance between these two lines is 0.2 mm. The hydrophilic coating used comprises an aqueouslethanolic solution (in a ratio of 4:1) composed of 0.5% by weight of Tegopren 5840 (surfactant based on an organomodified silicone having a polar polyether group) from Goldschmidt GmbH, 20% by weight of Luvitec K30 (polyvinylpyrrolidone with K value of 30) from BASF AG and 0.3% by weight of Irganox 1010 antioxidant from Ciba AG. The coating is dried thermally. The hydrophobic coating used comprises the UV release lacquer UVX00192 (UV-drying, cationic UV release lacquer) from XSys GmbH. The coating is cured by means of UV radiation.

In comparison with Inventive Example 1, the hydrophilic coating exhibits the same good wetting properties and similarly exhibits a high transport velocity of the aqueous test liquid in the test channel. Here again, liquid transport in the test channel reliably stops at the edge of the hydrophobic line. Nor does this transport behaviour of the test liquid alter when the test channels are stored for 6 weeks at 40° C. or 70° C. prior to the test.

### Comparative Examples

### Comparative Example 1

The corona-pretreated Hostaphan® RN 100 PET film from Mitsubishi Polyesterfilm GmbH of thickness 100 μm is printed, as in Inventive Example 2, by the flexographic printing process with a hydrophilic line of width 1.0 mm. The hydrophilic coating used comprises an aqueous/ethanolic solution (in a ratio of 4:1) composed of 0.5% by weight of Tegopren 5840 from Goldschmidt GmbH, 20% by weight of Luvitec K30 from BASF AG and 0.3% by weight of Irganox 1010 from Ciba AG. The coating is dried thermally.

The hydrophilic coating exhibits very good transport properties of the test liquid in the channel test. However, liquid transport does not stop at the end of the hydrophilic line. The test liquid is transported as far as the end of the test channel. Even enlargement of the width to 5 cm does not stop liquid transport but only slows it.

### Comparative Example 2

The corona-pretreated Hostaphan® RN 100 PET film from Mitsubishi Polyesterfilm GmbH of thickness 100 μm is printed by the flexographic printing process first with a hydrophobic line of width 2.0 mm and then with a hydrophobic line of width 1.0 mm. The two lines run parallel to one another, and the distance between these two lines is 0.2 mm. The hydrophilic coating used comprises a 20% strength by weight aqueous solution composed of Luvitec K30 from BASF AG. The coating is dried thermally. The hydrophobic coating used comprises UV release lacquer UVX00192 from XSys GmbH. The coating is cured by means of UV radiation.

The hydrophilic coating exhibits only moderate weighting properties, these being apparent in a relatively high wetting angle. There is only unreliable transport of the aqueous test liquid in the test channel. The start of transport into the channel is often delayed or fails to occur. If liquid transport into the channel occurs, great variation in transport velocity can be observed. However, this transport then reliably stops at the hydrophobic line.

### Comparative Example 3

The corona-pretreated Hostaphan® RN 100 PET film from Mitsubishi Polyesterfilm GmbH of thickness 100 μm is printed, as in Inventive Example 2, by the flexographic printing process first with a hydrophobic line of width 2.0 mm and then with a hydrophobic line of width 1.0 mm. The two lines run parallel to one another, and the distance between these two lines is 0.2 mm. The hydrophilic coating used comprises an aqueous/ethanolic solution (in a ratio of 4:1) composed of 0.5% by weight of Tegopren 5840 from Goldschmidt GmbH, 20% by weight of Luvitec K30 from BASF AG and 0.3% by weight of Irganox 1010 from Ciba AG. The coating is dried thermally. The hydrophobic coating used comprises UV printing ink UFZ 50129 from Siegwerk GmbH. The coating is cured by UV radiation.

The hydrophilic coating exhibits very good transport properties of the test liquid in the channel test. However, stoppage of liquid transport at the edge of the hydrophobic line is not achieved, all that occurs being brief slowing of liquid transport. The liquid front then breaks through and liquid is transported as far as the end of the test channel.

Overview of properties of inventive examples and comparative examples.

| | | | | | |
|---|---|---|---|---|---|
| | Inventive | Inventive | Comparative | Comparative | Comparative |
| Unit | Example 1 | Example 2 | Example 1 | Example 2 | Example 3 |

Hydrophilic region

| | | | | | | |
|---|---|---|---|---|---|---|
| Contact | ° | 21 | 19 | 19 | 58 | 19 |
| angle | | | | | | |
| Surface | mN/m | 67 | 69 | 69 | 57 | 69 |
| tension | | | | | | |

Hydrophobic region

| | | | | | | |
|---|---|---|---|---|---|---|
| Contact | ° | 102 | 109 | 73 (PET) | 109 | 82 |
| angle | | | | | | |
| Surface | mN/m | 27 | 24 | 45 (PET) | 24 | 37 |
| tension | | | | | | |
| Separation | cN/cm | 32 | 26 | — | 26 | 190 |
| value | | | | | | |

1. Sheet-like material comprising a substrate material on whose upper side at least one hydrophilic region and at least one hydrophobic region are present and arranged alongside one another, where
a contact angle of the hydrophilic region with water is smaller than 30° and
a contact angle of the hydrophobic region with water is greater than 90°.

2. Sheet-like material according to claim 1,
wherein
on the substrate material a plurality of hydrophilic and a plurality of hydrophobic regions are present arranged alongside one another.

3. Sheet-like material according to claim 1,
wherein
the hydrophilic region(s) is/are composed of a coating with
a surface tension of at least 63 mN/m and
a contact angle with water which is smaller than 25°.

4. Sheet-like material according to claim 1,
wherein
the hydrophilic region comprises at least one surfactant.

5. Sheet-like material according to claim 1,
wherein
the hydrophilic region is composed of at least one surfactant and of at least one polymer.

6. Sheet-like material according to claim 1,
wherein
the hydrophobic region(s) is/are composed of a coating with
a surface tension of at most 30 mN/m and
a contact angle with water which is greater than 95° and
a separation value smaller than 50 cN/cm in a test with an acrylate adhesive tape.

7. Sheet-like material according to claim 1,
wherein
the hydrophobic region is composed of a release lacquer based on a stearyl compound, on a fluoropolymer or on a silicone polymer.

8. Sheet-like material according to claim 1,
wherein
the hydrophobic region is composed of a UV-crosslinkable release lacquer.

9. Sheet-like material according to claim 1,
wherein
the hydrophilic and hydrophobic regions on the upper side of the substrate material are produced by partial application, to a full-surface coating of one of the regions, of the other region(s).

10. Sheet-like material according to claim 1,
wherein
the hydrophilic and/or the hydrophobic region are applied by means of a doctor process, a spray process or a printing process to the substrate material.

11. Diagnostic strips, biosensors, point-of-care devices or a microfluidic device.

12. Biosensors or analytical test strips comprising sheet-like material according to claim 1, where an arrangement of the material is such that the upper side of the substrate material forms the wall of a transport channel or of a reaction channel.
